(19) United States
(12) Patent Application Publication
(10) Pub. No.: US 2015/0374938 A1
(43) Pub. Date: Dec. 31, 2015

(54) ACTIVE INGREDIENT DEPOT FOR AN INHALATION DEVICE

(71) Applicant: SIMILTRADE GMBH, Rotkreuz (CH)

(72) Inventors: Patrik SCHEIBER, Niederhasli (CH); Anton SPOERRI, Weggis (CH); Markus SPOERRI, Unteraegeri (CH)

(73) Assignee: Similtrade GMBH, Rotkreuz (CH)

(21) Appl. No.: 14/767,630

(22) PCT Filed: Feb. 14, 2014
(86) PCT No.: PCT/EP2014/000417

§ 371 Date: Aug. 13, 2015

(30) Foreign Application Priority Data
Feb. 14, 2013 (DE) 10 2013 002 581.8

(51) Int. Cl. A61M 15/00; A61M 15/06

(52) U.S. Cl. CPC A61M 15/0028; A61M 15/06; A61M 2205/3613

(57) ABSTRACT

An active substance depot for an inhalation device having a depot body externally sealed in an airtight manner prior to use, comprising at least one segment made from a plastic material, having a flow channel to be unsealed for use, and having at least one active substance carrier provided in the flow channel in which is stored at least one volatile substance at least partially transitioning into the gaseous phase when heated, the active substance carrier being heated for releasing the substance. According to the invention, the plastic material of which the segment of the depot body is made is a polymer from the group of cyclo olefin copolymers, and the segment of the depot body made from the polymer comprises an opening connected to the flow channel, said opening being sealed by a foil made of a second material, and said foil being removed or punctured immediately prior to use.

The invention relates to an active substance depot for an inhalation device having a depot body externally sealed in an airtight manner prior to use, comprising at least one made from a plastic material, having a flow channel to be unsealed for use, and having at least one active substance carrier provided in the flow channel in which is stored at least one volatile substance at least partially transitioning into the gaseous phase when heated, the active substance carrier being heated for releasing the substance.

For some time, inhalation devices by means of which users can inhale volatile substances have been in use. The inhalation device comprises an active substance depot to this end, from which the substance is released to an airflow drawn through the inhalation device by the user.

Such inhalation devices are used, among other purposes, for inhaling pharmaceutically active substances, such as for treating throat and neck pain, or for treating diseases of the lungs. The advantage thereby is that a defined and very targeted delivery of the volatile substances serving as active substances is possible by relatively simple technical means. In order to eliminate or alleviate any unpleasant side effects, it is also possible to store other substances in addition to the pharmaceutically active substances, such as pain relieving active substances, flavoring agents, and the like, which are released simultaneously with the pharmaceutically active substance.

Instead of pharmaceutically active substances, the active substances can also be nicotine or compounds comprising nicotine to be consumed by the user. The relevant advantage therein lies in the fact that the consumption can take place without tobacco having to be burned in order to produce smoke. Said so-called smokeless cigarettes, cigars, or pipes are designed as inhalation devices by means of which active substances such as nicotine or compounds comprising nicotine, as well as flavoring agents, can be released in a targeted and defined manner and then inhaled by the user. The term “compound comprising nicotine” is understood in the present context to mean in particular nicotine salts, nicotine derivatives, and tobacco components comprising nicotine.

Depending on the type of inhalation device, the active substance depot is either a substantial component of the inhalation device and is disposed of along with the same when no more active substance is stored in the active substance depot, or the active substance depot is designed as a disposable depot that is placed in a reusable inhalation device and is then removed from the inhalation device and disposed of after use.

The active substance depots are typically produced centrally in very large quantities and then shipped. Therefore the active substance depots must be designed so as to be safely transported and stored without the active substances stored in the active substance depot being released during transport or storage.

In order to ensure this, it is therefore typical to use materials for the depot body of the active substance depot that are sufficiently gas-tight and prevent diffusion of the active substance through the material out of the active substance depot. It is furthermore typical to also pack the active substance depots in blister packages in order to additionally minimize the release of active substances.

The object of the present invention is thus to provide an active substance depot that can be produced in very large quantities and simultaneously is externally sealed so that unintended release of active substances is effectively prevented.

The object is achieved according to the invention by an active substance depot having the features according to claim 1, and particularly in that the plastic material of which the segment of the depot body is made is a polymer from the group of cyclo olefin copolymers, and that the segment of the depot body made from the polymer comprises an opening connected to the flow channel, said opening being sealed by a foil made of a second material, and said foil being removed or punctured immediately prior to use.

The invention relates to the fact that, from a nearly infinite number of potential materials, a plastic material comprising a wide variety of properties, even somewhat conflicting properties, is selected.

The plastic should be easy to process with a low level of technical effort, while not only being resistant to the substances stored in the active substance carrier, but also effectively preventing unintended diffusion out of the active substance depot of the substances stored in the active substance depot.

Plastic materials that are easily processed, that is, thermoplastics suitable for injection molding, have long been known. The known injection-moldable plastics, however, are characterized in that said plastics have only limited resistance to particularly substances, or even if said plastics are resistant, said plastics comprise too great a water vapor permeability, so that said plastics are not normally selected for use in the active substance depot according to the invention.

Plastics that have, in contrast, sufficient resistance and as low a substance permeability as possible, are also known, but are typically difficult to process and are particularly ill suited for injection molding.

A third criterion to be considered for selecting a suitable plastic material is that the active substance depot must be sealed in a gas-tight manner after filling. The sealing must thereby take place such that an absolutely gas-tight bond is ensured between the plastic material of the depot body and the sealing material. Independently thereof, the effects and loads on the active substance depot arising during sealing, such as temperature effects when heat sealing, must be so moderate that the highly volatile substances already present in the active substance depot prior to sealing are not unintentionally released by the sealing process and do not leak out at least partially while sealing.

The various objects are achieved according to the invention in that a polymer from the group of cycle olefin copolymers is selected as the plastic material for the active substance depot.

Said polymer is easy to process, such as by injection molding, so that large quantities can be produced in a very simple manner at moderate cost and having complex geometries. The polymer is simultaneously resistant to a plurality of substances, however, and comprises an extremely low water vapor permeability.

A further surprising advantage lies in the fact that the polymer can be bonded in a gas-tight manner to suitable foil materials with little effort and very slight effects and loads on the polymer, contrary to the common opinion among technical experts.

Only the combination of said three substantial properties makes it possible at all to produce active substance depots in sufficient numbers that are not only resistant to the substances store in the active substance depots and comprise negligible water vapor permeability, but also can be sealed without great effort after being filled with the volatile substances.

For the active substance depot according to the invention, it is proposed that at least the segment of the depot body comprising the opening to be sealed, said opening being connected to the flow channel, is manufactured from a polymer from the group of cyclo olefin copolymers. The polymer is not only resistant to a plurality of volatile substances, but also comprises a relative low permeability to water vapor and can be surprisingly well sealed by means of foils. Prior to use the foil must merely be removed or punctured, wherein the term “punctured” can also of course be understood to mean pierced, penetrated, or lanced, with or without the use of a tool.

The polymer from the group of cyclo olefin copolymers is further suitable for injection molding. The segment of the depot body made from the cyclo olefin copolymer is therefore preferably an injection molded part.

Further advantages of the invention are found in the following description, the subclaims, and the drawings.

In order to prevent as much as possible the volatile substance stored in the active substance depot from diffusing out of the depot body, the permeability to water vapor of the polymer should be as low as possible. For this reason it is proposed that a polymer is used for the active substance depot comprising a water vapor permeability according to DIN 53122-2 (at an ambient temperature of 23° C. and relative humidity of 85%) in a range from 0.02 to 0.05 g mm/m2 d, preferably in a range from 0.03 to 0.04 g mm/m2 d, particularly preferably of 0.035 g mm/m2 d.

In order that a plurality of volatile substance having completely different chemical behavior can be stored in the active substance depot, a polymer from the group of cyclo olefin copolymers and resistant to hydrolysis, acids, bases, and polar organic solvents should be used for the depot body.

In order to enable trouble-free further processing of the segment of the depot body made from the polymer, it is further proposed that a polymer from the group of cyclo olefin copolymers is used, said polymer not tending to fracture in a brittle manner, but rather having sufficiently high toughness. For this reason it is proposed that a polymer is used having an impact strength within a range from 10 to 25 kJ/m2, preferably in a range from 15 to 20 kJ/m2, particularly preferably of 15 kJ/m2, according to ISO 179/1eU (test body 80 mm×10 mm×4 mm, narrow side impact direction, unnotched test body).

In order to minimize the effects of small recesses, notches, or surface damage, a polymer should be used having an impact strength within a range from 3.0 to 1.0 kJ/m2, preferably in a range from 1.6 to 2.6 kJ/m2, particularly preferably of 1.8 kJ/m2, according to ISO 179/1eU (test body 80 mm×10 mm×4 mm, narrow side impact direction, test body notched with 0.25 mm radius).

A further essential concept of the invention relates to the use of a foil by means of which the opening in the segment of the depot body made from the polymer is to be sealed. Contrary to the opinion of persons skilled in the art, a metal foil is used and bonded to the perimeter of the opening in the depot body in a gas-tight manner by means of heat sealing, wherein the polymer, contrary to assumption, takes on a gas-tight and close bond with the heat sealing lacquer coating of the metal foil. The opening in the depot body is well sealed externally in this manner, wherein the level of heating arising from heat sealing is so low that unintended release of volatile substance from the active substance carrier does not occur. Tests have shown that the seal can resist pressure differentials of up to 0.3 bar without damage.

It is particularly advantageous for said embodiment to use to use a metal foil, preferably an aluminum foil, having a material thickness in a range from 20 to 40 μm, preferably a material thickness in a range from 25 to 35 μm, particularly preferably a material thickness of 30 μm, while the flat side of the foil sealed to the depot body has a heat sealing lacquer coating by means of which the metal foil is bonded in a gas-tight manner to the polymer of the segment of the depot body.

Due to the relatively low material thickness, heat can be transferred quickly and in a targeted manner to the heat sealing lacquer coating to be fused to the depot body, so that the sealing of the opening takes place very quickly and without great heating effects on the depot body. At the same time, the polymer of which the depot body is made at least in segments, has a heat insulating effect, so that the heat energy introduced during heat sealing is not transferred to the active substance carrier and thus unintended release of the volatile substances from the active substance carrier is prevented.

For a particularly preferred embodiment of the active substance depot according to the invention, the depot body comprises two segments made of the polymer, between which a metal sleeve is disposed, the free ends thereof being connected in a gas-tight manner to the segments made of the polymer. The at least one active substance carrier in which the volatile substances are stored is retained in the metal sleeve. The metal sleeve acts as a heat transfer element and transfers the body heat of the user to the active substance carrier during use, so that the substance stored in the active substance carrier is released to the air drawn in by the user through the flow channel.

Said preferred embodiment has the advantage that the centered arrangement of the metal sleeve between the segments made of polymer causes the heat to be transferred to the active substance carrier in a very targeted manner, while the segments made of polymer have a thermally insulating effect and thus at least a substantial part of the heat energy introduced via the metal sleeve is transferred to the active substance carrier.

Said active substance depot particularly provided for passive inhalation devices has the advantage that the heat required for releasing the volatile substance is introduced into the active substance depot only via the body heat of the user. During use the user holds the active substance depot in the externally accessible region of the metal sleeve and the body temperature of the user transferred via the fingers of the user heats the substances stored in the active substance carrier. The thermal conductivity and heat capacity of the metal sleeve are thereby designed so that the body temperature of the user is sufficient for heating the active substance carrier to the extent that the substances stored in the active substance carrier are released. Because the active substance carrier is disposed in the flow channel of the active substance depot through which the user draws air, the substances released from the active substance carrier are introduced into the air flow.

For a particularly preferred variant of said embodiment, the two segments of the depot body made from the polymer each have an opening connected to the flow channel, each being sealed by a sealing foil bonded to the respective segment of the depot body by means of heat sealing, said foil being removed or pierced immediately prior to use.

The invention is described in more detail below, using an embodiment example and referencing the figures. They show:

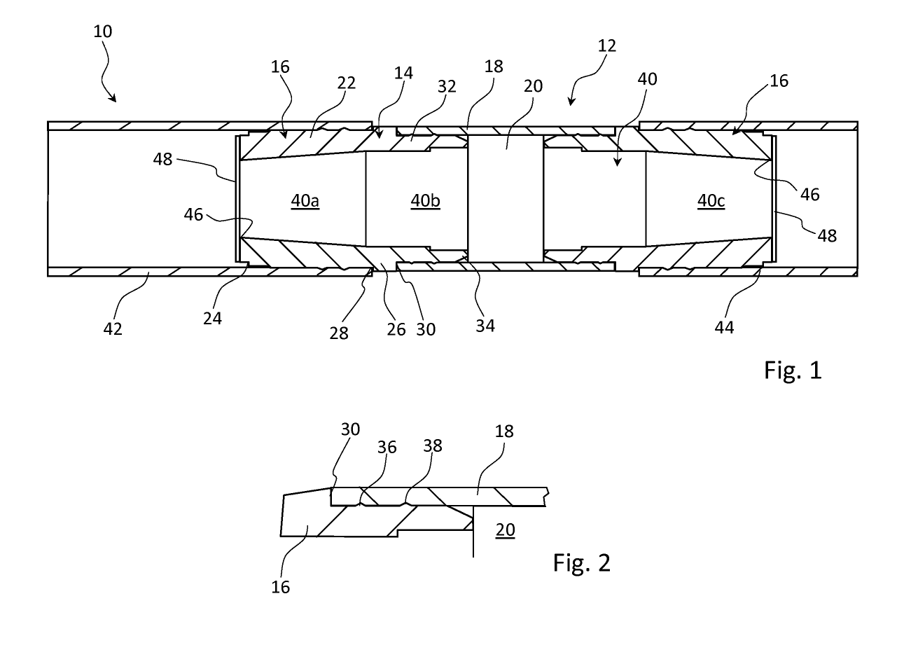

FIG. 1 A section view through an embodiment example of an inhalation device having an active substance depot according to the invention, and

FIG. 2 An enlarged section from FIG. 1 wherein the transition between a metal sleeve and a sleeve made of a polymer is shown.

FIG. 1 shows a section view of an embodiment example of an inhalation device 10 comprising an active substance depot 12 according to the invention.

The active substance depot 12 comprises a depot body 14. The depot body 14 has two identically implemented sleeves 16 between which a metal sleeve 18 is disposed. The metal sleeve 18 is implemented in the form of a hollow cylinder and made of a material having very high thermal conductivity, such as of aluminum or of an aluminum alloy.

An active substance carrier 20 is received in the metal sleeve 18, in which is stored at least one substance volatile at room temperature, such as an active substance to be released, a flavoring agent, or an aroma agent. The active substance carrier 20 is implemented in the shape of a cylinder and makes contact with the inner surface of the metal sleeve 18. In the present case the active substance carrier 20 is used for storing a specified amount of a pharmaceutically active substance.

The average draw volume of 35 ml and the draw time of an average user of 1 to 2 seconds result in a very brief contact time between the air flowing through the active substance carrier 20 and the pharmaceutically active substance stored in the active substance carrier 20. The transition of the substance to be released into the gas phase correlates approximately to the contact surface between the active substance carrier 20 and the air.

The active substance carrier 20 shown is received in the metal sleeve 18 so that the metal sleeve 18 makes direct contact with the hand of the user when the inhalation device 10 is held. The heat of the hand is then transferred to the metal sleeve 18, and in turn the heat is thereby conducted further inward to the active substance carrier 20. The heating of the active substance carrier 20 increases the release rate of the volatile substance and further substances optionally stored in the active substance carrier 20, such as flavoring agents, such that a sufficient amount of the volatile substance and the flavoring agent can be received by the user with each draw. The volume of the sleeve 18 is adapted to the wall thickness thereof so that the heating time after positioning between the fingers runs as quickly as possible.

One of the two sleeves 16 made of plastic is inserted in each open end of the metal sleeve 18. The two sleeves 16 are implemented identically, so that consequently only the sleeve 16 shown on the left in FIG. 1 is described in further detail.

The rotationally symmetrical sleeve 16 has a segment 22 of medium external diameter at the end thereof shown on the left in FIG. 1. The end of the segment 22 shown on the left in FIG. 1 has a chamfer 24.

A peripheral flange 26 of greater diameter and extending radially outward is connected to the segment 22, said segment extending over about two-thirds of the axial length of the sleeve 16. The flange 26 has a first contact surface 28 as seen in the axial direction and shown on the left in FIG. 1 and a second contact surface 30 shown on the right in FIG. 1.

The second contact surface 30 of the flange 26 transitions into an offset 32 of smaller diameter. The free end of the offset 32 also has a chamfer 34. The offset 32 of the sleeve 16 is pressed into the hollow cylindrical metal sleeve 18 until the end face of the metal sleeve 18 makes contact with the second contact surface 30 of the flange 26. The outside diameter of the offset 32 is thereby dimensioned so that the offset 32 forms a press fit with the inner peripheral surface of the metal sleeve 18.

FIG. 2 shows an enlarged detail from FIG. 1. As can be seen in FIG. 2, the offset 32 of the sleeve 16 additionally has two peripheral sealing flanges 36 and 38 making contact under initial tension with the inner peripheral surface of the metal sleeve 18 in order to connect the metal sleeve 18 to the sleeve 16 in a gas-tight manner. The identically implement sleeve 16 shown on the right in FIG. 1 and facing to the left is correspondingly connected to the metal sleeve 14 in a gas-tight manner.

The axial length of the offset 34 of each sleeve 16 is selected and matched to the axial length of the depot 18 so that the depot 18 is restrained and centered in the heat transfer sleeve 14 between the offsets 34 of the two sleeves 16 when the inhalation device 10 is assembled, but at least disposed between said sleeves.

A flow channel 40 also extends through the inhalation device 10. The flow channel 40 is subdivided into a plurality of segments 40a through 40c, namely a first segment 40a implemented in the first sleeve 16 and expanding in the shape of a hollow cone, a second segment 40b implemented in the metal sleeve 18, in which the active substance carrier 20 is received, and a third segment 40c implemented in the second sleeve 16 and tapering in the shape of a hollow cone.

An intake tube 42 made of paper is placed on the free end of the segment 22 of medium diameter of the sleeve 16 shown on the left in FIG. 1. A mouthpiece 44 made of paper is placed on the free end of the segment 22 of medium diameter of the sleeve 16 shown on the right in FIG. 1. Both the intake tube 42 and the mouthpiece 44 are also implemented as hollow cylinders and are connected to the flow channel 40.

The two identically designed sleeves 16 are implemented as injection molded parts and are made of a polymer from the group of cyclo olefin copolymers. The polymer used for the two sleeves 16 is resistant to hydrolysis, acids, bases, and polar organic solvents and has a water vapor permeability in a range from 0.03 to 0.04 g mm/m2 d. The water vapor permeability has been determined according to DIN 53122-2 at an ambient temperature of 23° C. and relative humidity of 85%.

As can further be seen in FIG. 1, the opening 46 at the free end of each sleeve 16 is sealed by a metal foil 48. The metal foil 48 is made of aluminum and has a material thickness of 30 μm. The metal foil 48 has a heat sealing lacquer coating on the flat side thereof facing toward the sleeve 16, by means of which the metal foil has been bonded to the sleeve in a gas-tight manner by heat sealing.

In order to use the inhalation device 10, the metal foil 48 must simply be punctured or pierced so that the user can draw air through the flow channel 40.

### LIST OF REFERENCE NUMERALS

10 Inhalation device
12 Active substance depot
14 Depot body
16 Sleeves
18 Metal sleeve
20 Active substance carrier
22 Medium diameter segment
24 Chamfer
26 Flange
28 First contact surface
30 Second contact surface
32 Smaller diameter offset
34 Chamfer
36 Sealing joint
38 Sealing joint
40 Flow channel
40a First segment of the flow channel
40b Second segment of the flow channel
40c Third segment of the flow channel
42 Intake tube
44 Mouthpiece
46 Opening
48 Metal foil

1. An active substance depot for an inhalation device having a depot body externally sealed in an airtight manner prior to use, comprising at least one segment made from a plastic material, having a flow channel to be unsealed for use, and having at least one active substance carrier provided in the flow channel in which is stored at least one volatile substance at least partially transitioning into the gaseous phase when heated, the active substance carrier being heated for releasing the substance,
wherein
the plastic material of which the segment of the depot body is made is a polymer from the group of cyclo olefin copolymers, and that the segment of the depot body made from the polymer comprises an opening connected to the flow channel, said opening being sealed by a foil made of a second material, and said foil being removed or punctured immediately prior to use.

2. The active substance depot according to claim 1, wherein the polymer comprises a water vapor permeability according to DIN 53122-2 (at an ambient temperature of 23° C. and relative humidity of 85%) in a range from 0.02 to 0.05 g mm/m² d, preferably in a range from 0.03 to 0.04 g mm/m² d, particularly preferably of 0.035 g mm/m² d.

3. The active substance depot according to claim 1, wherein the plastic material of which the segment of the depot body is made is resistant to hydrolysis, acids, bases, and polar organic solvents.

4. The active substance depot according to claim 1, wherein the impact strength of the polymer of which the segment of the depot body is made is within a range from 10 to 25 kJ/m², preferably in a range from 15 to 20 kJ/m², particularly preferably of 15 kJ/m², according to ISO 179/1 eU (test body 80 mm×10 mm×4 mm, narrow side impact direction, unnotched test body).

5. The active substance depot according to claim 1, wherein the notch impact strength of the polymer of which the segment of the depot body is made is within a range from 3.0 to 1.0 kJ/m², preferably in a range from 1.6 to 2.6 kJ/m², particularly preferably of 1.8 kJ/m², according to ISO 179/1 eU (test body 80 mm×10 mm×4 mm, narrow side impact direction, test body notched with 0.25 mm radius).

6. The active substance depot according to claim 1, wherein the opening provided on the segment of the depot body made of the polymer is sealed gas-tight by means of a metal foil bonded to the polymer by hot sealing, said foil being removed or punctured immediately prior to use.

7. The active substance depot according to claim 6, wherein the metal foil, preferably an aluminum foil, comprises a material thickness in a range from 20 to 40 μm, preferably a material thickness in a range from 25 to 35 μm, particularly preferably a material thickness of 30 μm, and has a heat sealing lacquer coating on one of the flat sides thereof, by means of which the metal foil is bonded in a gas-tight manner to the polymer of the segment of the depot body.

8. The active substance depot according to claim 1, wherein the depot body comprises two segments made of the plastic material, between which a metal sleeve is disposed, the free end thereof being bonded to the segments in a gas-tight manner, that the at least one active substance carrier is secured in the metal sleeve, and that the metal sleeve acts as a heat transfer element and transfers the body heat of the user to the active substance carrier during use, such that the substance stored in the active substance carrier is released to the air drawn through the flow channel by the user.

9. The active substance depot according to claim 8, wherein the two segments of the depot body made of the plastic material each comprise an opening connected to the flow channel, each sealed by a sealing foil bonded to the depot body in a gas-tight manner by means of heat sealing, said foil being removed or punctured immediately prior to use.
